(19) United States
(12) Patent Application Publication
(10) Pub. No.: US 2018/0014821 A1
(43) Pub. Date: Jan. 18, 2018

(54) TENDON STRIPPER

(71) Applicant: ARTHREX, INC., Naples, FL (US)

(72) Inventors: Robert M. Weber, Chino Hills, CA (US); Jacob A. Jolly, Naples, FL (US)

(21) Appl. No.: 15/211,683

(22) Filed: Jul. 15, 2016

(51) Int. Cl. A61B 17/00

(52) U.S. Cl. CPC A61B 17/00008; A61B 17/00234; A61B 2017/00353; A61B 2017/00349

(57) ABSTRACT

A tendon stripper according to an exemplary aspect of the present disclosure includes, inter alia, a shaft including a tip tapering to a point at the distal end of the shaft, and a retention structure moveable between an open position and a closed position to capture and retain a tendon relative to the tip. The tip arrangement increases the ease of separating a tendon from adjacent muscle tissue.

### BACKGROUND

This disclosure relates to a tendon stripper for use in separating at least a portion of a tendon from adjacent muscle tissue.

Tendon strippers are used to harvest tendons for use in orthopedic procedures. In one known procedure, a tendon stripper is used to harvest at least a portion of a hamstring. The harvested hamstring tendon may then be used in an anterior cruciate ligament (ACL) reconstruction surgery, for example. Tendons are also known to be harvested from other areas of the body, such as the wrist, and can used in other types of orthopedic procedures, such as ulna collateral ligament (UCL) reconstructions.

### SUMMARY

This disclosure relates to a tendon stripper, which is used to separate at least a portion of a tendon from adjacent muscle tissue. The harvested tendon can then be used in various orthopedic procedures, such as ACL and UCL reconstructions, to name a few examples.

A tendon stripper according to an exemplary aspect of the present disclosure includes, inter alia, a shaft including a tip tapering to a point at the distal end of the shaft, and a retention structure moveable between an open position and a closed position to capture and retain a tendon relative to the tip. The tip arrangement increases the ease of separating a tendon from adjacent muscle tissue.

A tendon stripper according to another exemplary aspect of the present disclosure includes, inter alia, a shaft including a tip provided at a distal end of the shaft, and a retention structure moveable between an open position and a closed position to capture and retain a tendon relative to the tip. The retention structure is provided entirely in a common plane containing a centerline of the shaft. The retention structure arrangement provides the retention structure with symmetry, which makes the tendon stripper easy to use on either side of a patient's body, and by either hand of the user.

A method according to an exemplary aspect of the present disclosure includes, inter alia, capturing a tendon in a retention structure of a tendon stripper, and moving the tendon stripper distally to separate at least a portion of a tendon from adjacent muscle tissue. The tendon stripper includes a shaft with a tip at a distal end of the shaft. The tip tapers to a point, which increases the ease of separating tendon from adjacent muscle.

### BRIEF DESCRIPTION OF THE DRAWINGS

FIG. 1 illustrates an example tendon stripper with a retention structure in a closed position.

FIG. 2 illustrates the example tendon stripper with the retention structure in an open position.

FIG. 3 is a side view of the tendon stripper of FIGS. 1-2 with the retention structure in the open position, and illustrates the detail of the retention structure and a tip of the tendon stripper.

FIG. 4 is a perspective view of the tendon stripper of FIGS. 1-2, and illustrates the detail of the retention structure and the tip.

FIG. 5 is a side view of the tendon stripper of FIGS. 1-2 with the retention structure in the closed position, and illustrates the detail of the retention structure and the tip.

FIG. 6A is a cross-sectional view taken along line 6A-6A from FIG. 5, and illustrates a first example tip configuration.

FIG. 6B is a cross-sectional view taken along line 6B-6B from FIG. 5, and illustrates a second example tip configuration

FIG. 7 illustrates the tendon stripper of FIGS. 1-2 in use.

FIG. 8 is a side view of another example tendon stripper. In this example, the retention structure has a straight leading surface.

### DETAILED DESCRIPTION

This disclosure relates to a tendon stripper, which is used to separate one or more tendons from adjacent muscle tissue. A harvested tendon can then be used in various orthopedic procedures, such as ACL and UCL reconstructions, to name a few examples.

A tendon stripper according to an exemplary aspect of the present disclosure includes, inter alia, a shaft including a tip tapering to a point at the distal end of the shaft, and a retention structure moveable between an open position and a closed position to capture and retain a tendon relative to the tip. The tip arrangement increases the ease of separating a tendon from adjacent muscle tissue.

In a further embodiment, the tip tapers to a rounded point.

In a further embodiment, the tip tapers to a sharp point.

In a further embodiment, the point is coincident with a centerline of the shaft.

In a further embodiment, the retention structure is a hook.

In a further embodiment, the hook is provided entirely in a common plane containing a centerline of the shaft.

In a further embodiment, the tendon stripper includes a handle. The shaft projects distally from the handle, and the handle includes a trigger provided in the common plane. Further, movement of the trigger moves the hook between the open and closed positions.

In a further embodiment, movement of the trigger in the distal direction moves the hook to the open position.

A tendon stripper according to another exemplary aspect of the present disclosure includes, inter alia, a shaft including a tip provided at a distal end of the shaft, and a retention structure moveable between an open position and a closed position to capture and retain a tendon relative to the tip. The retention structure is provided entirely in a common plane containing a centerline of the shaft. The retention structure arrangement provides the retention structure with symmetry, which makes the tendon stripper easy to use on either side of a patient's body, and by either hand of the user.

In a further embodiment, the retention structure is a hook including a first linear portion, a second linear portion, and a curved portion connecting the first and second linear portions. The first and second linear portions are substantially parallel to one another along their respective lengths.

In a further embodiment, the first linear portion is at least partially received within the shaft and mechanically coupled to a trigger provided in a handle. Further, the shaft projects distally from the handle, and movement of the trigger moves the hook between the open position and the closed position.

In a further embodiment, the trigger is provided in the common plane.

In a further embodiment, the second linear portion has a proximal end configured to be received in a socket adjacent the tip when the hook is in the closed position.

In a further embodiment, the curved portion follows an arc defined by a circle having a center between the first and second linear portions. Further, the first and second linear portions are spaced-apart from one another by a distance substantially equal to a diameter of the circle along substantially their entire respective lengths.

In a further embodiment, the curved portion makes substantially a 180° bend about the center to connect the first and second linear portions.

In a further embodiment, the tip tapers to a point coincident with the centerline of the shaft.

In a further embodiment, the retention structure includes one of a hook, a square, a loop, a corkscrew, and a slotted sleeve.

In a further embodiment, the retention structure includes a straight leading edge.

A method according to an exemplary aspect of the present disclosure includes, inter alia, capturing a tendon in a retention structure of a tendon stripper, and moving the tendon stripper distally to separate at least a portion of a tendon from adjacent muscle tissue. The tendon stripper includes a shaft with a tip at a distal end of the shaft. The tip tapers to a point, which increases the ease of separating tendon from adjacent muscle.

In a further embodiment, the point of the tip is provided at a junction between the tendon and the muscle tissue during the separating step, and the point of the tip is coincident with a centerline of the shaft.

FIG. 1 illustrates an example tendon stripper 20. The tendon stripper 20 includes a handle 22, a shaft 24, and a retention structure. In the figures, the retention structure is a hook 26. This disclosure is not limited to tendon strippers with hooks, however, and extends to other types of retention structures such as squares, loops, corkscrews, and slotted sleeves, to name a few examples.

The shaft 24 projects from the handle 22 in a distal direction. The hook 26 is moveable relative to the shaft 24 between an open position (FIG. 2) in which a tendon 54 (FIG. 7) can be captured by the hook 26, and a closed position (FIG. 1) in which the captured tendon 54 is secured relative to a tip 28 of the shaft. As will be explained below, the tip 28 is configured to separate the tendon 54 from adjacent muscle tissue 56 (FIG. 7).

In this example, the hook 26 is selectively moveable between the open and closed positions by movement of a trigger 30 provided on the handle 22. In this example, the trigger 30 is configured for movement by a user's (e.g., a surgeon's) thumb. The trigger 30 is moveable in the distal and proximal directions to move the hook between the open and closed positions, respectively. The “proximal” and “distal” directions are labeled across the figures for purposes of explanation only. In some examples, the trigger 30 is moveable to intermediate positions between the open and closed positions to accommodate different tendon sizes, for example. Further, in one example, the trigger 30 is mechanically biased to the closed position.

While in the disclosed embodiment the hook 26 is moveable relative to the shaft 24, in other examples the shaft 24 could be moveable relative to the hook 26. In that case, the shaft 24 would be selectively moveable in response to movement of the trigger 30.

The trigger 30 in this example is provided on a superior, or top, surface 32 of the handle 22. The handle 22 may also include a second trigger 34 (shown in phantom in FIG. 1) on an inferior, or bottom, surface 36 of the handle 22. The first trigger 30 and the second trigger 34, if present, are both configured to selectively move the hook 26 between the open and closed positions. In one example, the second trigger 34 moves with the first trigger 30. Alternatively, the second trigger 34 could move independently of the first trigger 30.

The arrangement of the hook 26 allows for increased ease of capturing a tendon within the hook 26. Further, the hook 26 and tip 28 arrangement provides for increased functionality of the tendon stripper 20. Namely, the arrangement allows the tendon stripper 20 to be easily used on both sides of a patient's body, and by either hand of a user (e.g., a surgeon).

With reference to FIG. 3, in one example the hook 26 is substantially U-shaped. In particular, the hook 26 includes a first linear portion 38, a second linear portion 40, and a curved portion 42 between the first and second linear portions 38, 40. The first and second linear portions 38, 40 extend in a linear direction along substantially straight lines, and are substantially parallel to one another along their entire respective lengths D₁, D₂.

In this example, the first linear portion 38 has a length D₁ greater than the length D₂ of the second linear portion 40. Further, the first linear portion 38 is received at least partially inside the shaft 24, and is mechanically coupled to the trigger 30. The first linear portion 38 may be directly coupled to the trigger 30, or the tendon stripper 20 may include an intermediate actuator, such as a shaft or cable, configured to transmit force from the trigger 30 to the first linear portion 38.

The proximal end 44 of the second linear portion 40 is configured to be received in a socket 46 provided adjacent the tip 28. When in the open position, as shown in FIG. 3, a tendon can be captured within the hook 26 by virtue of the space between the proximal end 44 of the second linear portion 40 and the tip 28.

In this example, the curved portion 42 follows an arc defined by a circle having a center at an origin O₁ between the first and second linear portions 38, 40. The circle has a radius R₁ originating at origin O₁ and a diameter D₃ passing through origin O₁. The curved portion 42 provides a semi-circle, and in this example makes a 180° bend about the origin O₁ to connect the first and second linear portions 38, 40. Further, the first and second linear portions 38, 40 are spaced-apart from one another by a distance D₄ substantially equal to the diameter D₃. Spacing the first and second linear portions 38, 40 apart from one another by the distance D₄ increases the ease of capturing a tendon within the hook 26. Further, the tip 28 spans the entire distance D₄ between the first and second linear portions 38, 40, which increases the available cutting surface.

With reference to FIG. 4, the hook 26 is provided entirely in a common plane P, in one example, and the hook 26 is bisected by the common plane P. That is, the first and second linear portions 38, 40 and the curved portion 42 are entirely provided in a common plane P. The first and second linear portions 38, 40 and the curved portion 42 do not bend out of the common plane P in the side-to-side directions (e.g., in or out of the page, relative to FIG. 4). In one example, the common plane P contains a centerline 50 (FIGS. 6A-6B) bisecting the tendon stripper 20. This arrangement provides the hook 26 with symmetry, which makes the tendon stripper 20 easy to use on either side of a patient's body, and by either hand of the user. To this end, the trigger 30 and the second trigger 34 (if present) are also provided in the common plane P such that the triggers 30, 34 are easily accessed by the user when the tendon stripper 20 is held in various orientations.

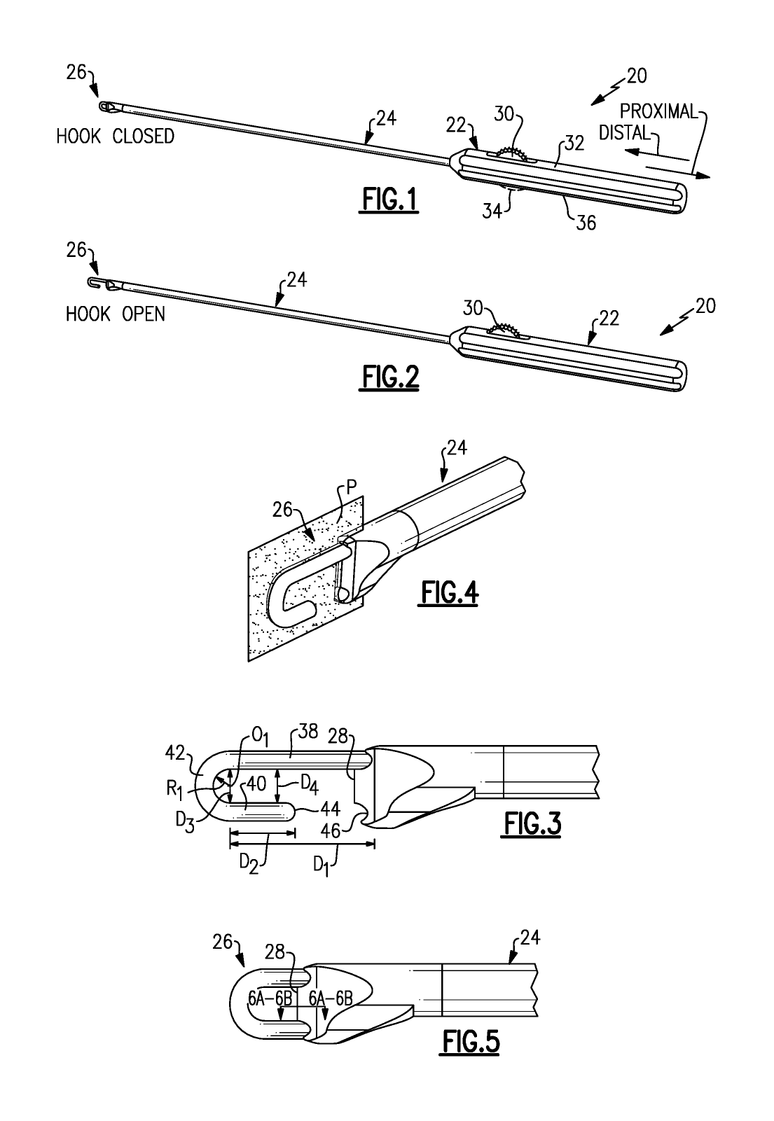

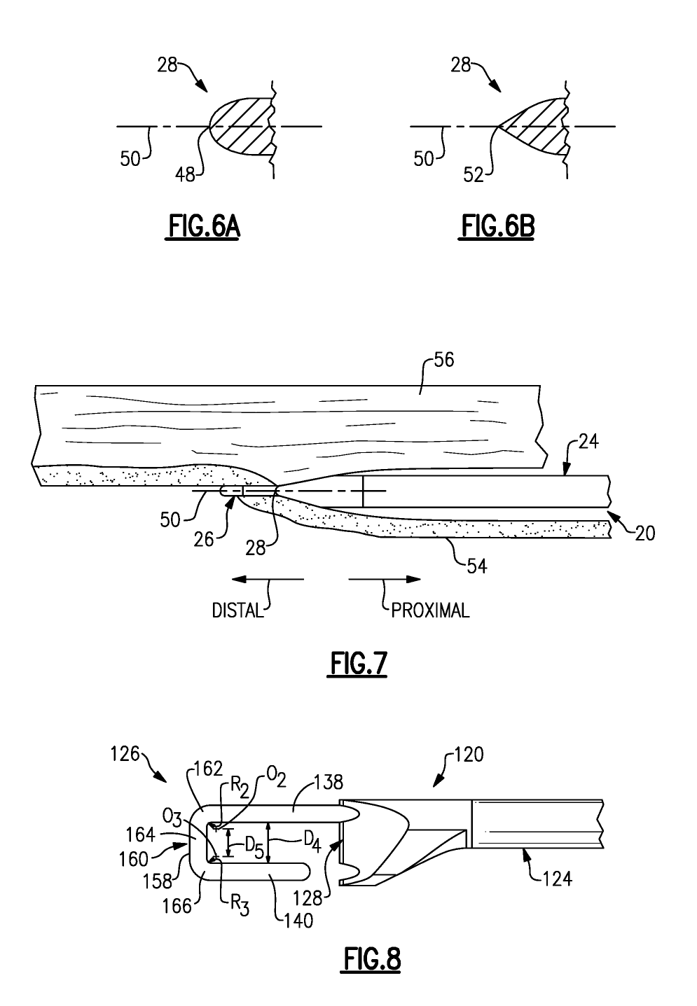

FIG. 5 illustrates the hook 26 in a closed position, in which the hook 26 and the tip 28 enclose a space for receiving a tendon 54. In one example, the tip 28 is provided along a centerline 50 (FIGS. 6A-6B) of the shaft 24. Additionally, the tip 28 may taper to a point. The tip 28 thus increases the ease of separating a tendon from adjacent muscle tissue.

In one example, illustrated in FIG. 6A, the tip 28 tapers to a rounded point 48. The rounded point 48 is provided on a centerline 50 of the shaft 24. The centerline 50 of the shaft 24 in one example is also a centerline of the tendon stripper 20, and is contained within the common plane P. In another example, the tip 28 tapers to a sharp point 52 (FIG. 6B). While the rounded point 48 is blunt (i.e., not sharp), the tip 28 still increases the ease of separating tendon from adjacent muscle tissue because the tip 28 is provided along the centerline 50.

FIG. 7 schematically illustrates the tendon stripper 20 in use. In FIG. 7, the hook 26 has captured a tendon 54. A user separates the tendon 54 from adjacent muscle tissue 56 by movement of the tendon stripper 20 in the distal direction. As shown in FIG. 7, the arrangement of the hook 26 and tip 28 is such that the point (e.g., points 48, 52) of the tip 28 is provided at a junction between the tendon 54 and the muscle tissue 56. With reference to the orientation of FIG. 7, the tendon 54 is below the centerline 50 proximal of the tip 28, and the muscle tissue 56 is above the centerline 50 proximal of the tip 28. The hook 26 of the present disclosure is configured to maintain the position of the tip 28 at the junction between the tendon 54 and the muscle tissue 56, while also allowing for ease of movement of the tendon stripper 20 in the distal direction.

FIG. 8 is a side view of another example tendon stripper 120. To the extent not otherwise described or shown, the tendon stripper 120 corresponds to the tendon stripper 20 of FIGS. 1-7, with like parts preappended with a “1.”

In FIG. 8, the tendon stripper 120 includes a retention structure with a straight leading edge 158. In this example, the retention structure is a hook 126. Again, this disclosure is not limited to hooks and extends to other types of retention structures.

The hook 126 in this example includes first and second linear portions 138, 140, which are substantially similar to the first and second linear portions 38, 40 of the hook 26. In the hook 126, the first and second linear portions 138, 140 are connected by a connection section 160 having a first curved portion 162, a straight portion 164 providing the straight leading edge 158, and a second curved portion 166.

In this example, the first curved portion 162 extends between the first linear portion 138 and the straight portion 164. The first curved portion 162 follows a radius R₂ of curvature. The radius R₂ extends from an origin O₂ between the first and second linear portions 138, 140, but substantially closer to the first linear portion 138 than the second linear portion 140. In this example, the first curved portion 162 bends 90° about the origin O₂.

The straight portion 164 in this example extends substantially perpendicular to the first and second linear portions 138, 140. The straight portion 164 provides the tendon stripper 120 with a straight leading edge 158, which facilitates movement of the tendon stripper 120 within a patient's body.

The second curved portion 166 connects the straight portion 164 and the second linear portion 140. The second curved portion 164 is shaped similar to the first curved portion 162, and in particular follows a radius R₃ of curvature, which extends from an origin O₃. The origin O₃ is between the first and second linear portions 138, 140, but is substantially closer to the second linear portion 140 than the first linear portion 138. In this example, the second curved portion 166 bends 90° about the origin O₃.

In this example, the straight portion 164 extends a distance D₅ between the first and second curved portions 162, 166. The distance D₅ is equal to the distance D₄ less the radiuses R₁, R₂. Further, the entirety of the hook 126 is provided in a common plane in the same way as the hook 26. The hook 126 provides the tendon stripper 120 with similar benefits to the hook 26, including increasing the ease of capturing a tendon, increasing ease of use when the tendon stripper is held in various positions, maintaining tip position during cutting, etc. While two hook arrangements have been illustrated across FIGS. 1-8, this disclosures extends to other hook arrangements.

It should be understood that terms such as “distal,” “proximal,” “superior,” “inferior,” etc., have been used herein for purposes of explanation, and should not be considered otherwise limiting. Terms such as “generally,” “substantially,” and “about” are not intended to be boundaryless terms, and should be interpreted consistent with the way one skilled in the art would interpret those terms.

Although the different examples have the specific components shown in the illustrations, embodiments of this disclosure are not limited to those particular combinations. It is possible to use some of the components or features from one of the examples in combination with features or components from another one of the examples.

One of ordinary skill in this art would understand that the above-described embodiments are exemplary and non-limiting. That is, modifications of this disclosure would come within the scope of the claims. Accordingly, the following claims should be studied to determine their true scope and content.

1. A tendon stripper, comprising:
a shaft including a tip tapering to a point at the distal end of the shaft; and
a retention structure moveable between an open position and a closed position to capture and retain a tendon relative to the tip.

2. The tendon stripper as recited in claim 1, wherein the tip tapers to a rounded point.

3. The tendon stripper as recited in claim 1, wherein the tip tapers to a sharp point.

4. The tendon stripper as recited in claim 1, wherein the point is coincident with a centerline of the shaft.

5. The tendon stripper as recited in claim 1, wherein the retention structure is a hook.

6. The tendon stripper as recited in claim 5, wherein the hook is provided entirely in a common plane containing a centerline of the shaft.

7. The tendon stripper as recited in claim 6, further comprising a handle, the shaft projecting distally from the handle, wherein the handle includes a trigger provided in the common plane, and wherein movement of the trigger moves the hook between the open and closed positions.

8. The tendon stripper as recited in claim 7, wherein movement of the trigger in the distal direction moves the hook to the open position.

9. A tendon stripper, comprising:
a shaft including a tip provided at a distal end of the shaft; and
a retention structure moveable between an open position and a closed position to capture and retain a tendon relative to the tip, wherein the retention structure is provided entirely in a common plane containing a centerline of the shaft.

10. The tendon stripper as recited in claim 9, wherein the retention structure is a hook including a first linear portion, a second linear portion, and a curved portion connecting the first and second linear portions, wherein the first and second linear portions are substantially parallel to one another along their respective lengths.

11. The tendon stripper as recited in claim 10, wherein the first linear portion is at least partially received within the shaft and mechanically coupled to a trigger provided in a handle, the shaft projecting distally from the handle, wherein movement of the trigger moves the hook between the open position and the closed position.

12. The tendon stripper as recited in claim 11, wherein the trigger is provided in the common plane.

13. The tendon stripper as recited in claim 11, wherein the second linear portion has a proximal end configured to be received in a socket adjacent the tip when the hook is in the closed position.

14. The tendon stripper as recited in claim 10, wherein the curved portion follows an arc defined by a circle having a center between the first and second linear portions, and wherein the first and second linear portions are spaced-apart from one another by a distance substantially equal to a diameter of the circle along substantially their entire respective lengths.

15. The tendon stripper as recited in claim 14, wherein the curved portion makes substantially a 180° bend about the center to connect the first and second linear portions.

16. The tendon stripper as recited in claim 9, wherein the tip tapers to a point coincident with the centerline of the shaft.

17. The tendon stripper as recited in claim 9, wherein the retention structure includes one of a hook, a square, a loop, a corkscrew, and a slotted sleeve.

18. The tendon stripper as recited in claim 9, wherein the retention structure includes a straight leading edge.

19. A method, comprising:
capturing a tendon in a retention structure of a tendon stripper; and
moving the tendon stripper distally to separate at least a portion of a tendon from adjacent muscle tissue, the tendon stripper including a shaft with a tip at a distal end of the shaft, wherein the tip tapers to a point.

20. The method as recited in claim 19, wherein the point of the tip is provided at a junction between the tendon and the muscle tissue during the separating step, and wherein the point of the tip is coincident with a centerline of the shaft.
